(19) United States
(12) Patent Application Publication
(10) Pub. No.: US 2023/0140419 A1
(43) Pub. Date: May 4, 2023

(54) SYSTEM FOR THE AUTOMATIC EVALUATION OF COGNITION AND CONSCIOUSNESS OF AN INDIVIDUAL THROUGH EXTERNAL STIMULATIONS

(71) Applicants: ICM (INSTITUT DU CERVEAU ET DE LA MOELLE ÉPINIÈRE), Paris (FR); INSERM (INSTITUT NATIONAL DE LA SANTÉ ET DE LA RECHERCHE MÉDICALE), Paris Cedex 13 (FR); CENTRE NATIONAL DE LA RECHERCHE SCIENTIFIQUE, Paris (FR); APHP (ASSISTANCE PUBLIQUE - HÔPITAUX DE PARIS), Paris (FR); SORBONNE UNIVERSITE, Paris (FR)

(72) Inventors: Federico RAIMONDO, Embourg (BE); Jacobo SITT, Paris (FR); Lionel NACCACHE, Paris (FR)

(73) Assignees: ICM (INSTITUT DU CERVEAU ET DE LA MOELLE ÉPINIÈRE), Paris (FR); INSERM (INSTITUT NATIONAL DE LA SANTÉ ET DE LA RECHERCHE MÉDICALE), Paris Cedex 13 (FR); CENTRE NATIONAL DE LA RECHERCHE SCIENTIFIQUE, Paris (FR); APHP (ASSISTANCE PUBLIQUE - HÔPITAUX DE PARIS), Paris (FR); SORBONNE UNIVERSITE, Paris (FR)

(21) Appl. No.: 17/766,311

(22) PCT Filed: Oct. 2, 2020
(86) PCT No.: PCT/EP2020/077729

§ 371 Date: Apr. 4, 2022

(30) Foreign Application Priority Data
Oct. 4, 2019 (EP) 19306283.3

(51) Int. Cl. A61B 5/383; A61B 5/00

(52) U.S. Cl. CPC A61B 5/383; A61B 5/7475; A61B 5/4076; A61B 2560/0223; A61B 2560/0475

(57) ABSTRACT

A system for automatic evaluation of cognition and consciousness of a subject, the system including a micro-controller, a digital-to-analog converter and at least one sensory stimulation element, wherein the system is configured to receive as input an information concerning a stimulation paradigm and to generate as output a sensory stimulation according to the stimulation paradigm and transmit at least one synchronization signal determined by the stimulation paradigm to a device for the acquisition of a physiological signal so as to synchronize the system for stimulation with the device for the acquisition of a physiological signal during an acquisition.

### FIELD OF INVENTION

The present invention pertains to the field of physiological signal evaluation. In particular, the present invention relates to the field of evaluation of cognitive function in communicant or non-communicating patients through analysis of at least one physiological signal during patient stimulation.

### BACKGROUND OF INVENTION

Advances in modern intensive care medicine have led to an increasing number of patients who survive critical illness and especially severe brain injuries. Part of these patients will regain consciousness after a short period in coma but others will remain in certain conditions grouped under the name of Disorders of Consciousness (DoC). Patients with disorders of consciousness are characterized by preserved wakefulness in the absence of clear evidence of awareness such that they remain unable to communicate with their surroundings.

For example, patients in a vegetative state/unresponsive wakefulness syndrome (VS/UWS) open their eyes, but they do not show conscious responses to sensory stimulation. When patients exhibit signs of fluctuating yet reproducible remnants of nonreflex behavior, such as visual pursuit, they are considered to be in a minimally conscious state (MCS). The diagnostic assessment of patients with disorders of consciousness is mainly based on the observation of motor and oculomotor behaviors at the bedside. The evaluation of nonreflex behavior, however, is not straightforward, as patients can fluctuate in terms of vigilance, and may suffer from cognitive and/or sensory impairments, from small or easily exhausted motor activity and pain, which may lead in the underestimation of the level of consciousness. In this discussion, an accurate assessment of consciousness and of consciousness recovery probability are of tremendous practical and ethical value. In addition to the prognosis issue, misdiagnosis of consciousness can have other dramatic consequences such as inadequate pain management or insufficient/inappropriate communication with an alleged unconscious patient.

Although it is acknowledged that clinical examination is the basis of any neurological assessment, it is also known that it is usually not sufficient. Misdiagnosis can have important consequences, such as inadequate pain management, prognosis underestimation and even improper end-of-life decisions. Moreover, even when correctly performed, diagnosis based in behavior only is not fully accurate and might erroneously label some patients that might have covert awareness.

There are also others pathologies that require an evaluation of consciousness (conscious state and conscious access) and of cognitive abilities such as all forms of peripheral or central locked-in syndromes, in psychiatric and neurologic forms of catatonia or in major types of apathy (e.g.: severe depression).

Previous studies showed that neuroimaging and neurophysiological tools suggest relatively accurate patient diagnosis and prediction of clinical outcome.

However, neuroimaging, such as functional MRI and neurophysiological techniques are generally complex to implement, especially on patients that are in a vegetative state/unresponsive wakefulness syndrome or minimally conscious state. When using neurophysiological techniques, the neurophysiological signal may be acquired on site without need to move the patient in order to by provide sensory stimulation to the patients. However, due to level of accuracy needed to this task, the neurophysiological signal has to be acquired with high spatial density (i.e. high density EEG), producing a large amount of raw data which requires important calculation power and therefore, data cannot usually be analyzed on site.

The sensory stimulation is configured to induce a cognitive process in the stimulated subject. The human brain has the ability to extract patterns or regularities in its environment, e.g. object A is always followed by object B but never by object C. The brain can detect transitional probabilities in an automatic way, i.e. even when the subject's attention is distracted, or when stimuli are presented below the threshold of awareness. Automatic brain responses to a violation of a rule (or regularity) can also be detected if the stimuli are in close or local temporal vicinity (i.e. within few seconds). Mismatch responses can be produced with complex sequences such as a melody or a rhythm even in unconscious subjects and are measurable on electroencephalographic data as event-related potentials (ERP). Previous studies have used Macintosh or Windows PC running customizable software to present various stimuli to the patients. Such desktop computers are computationally powerful and can present a variety of highly controlled and accurate stimulate, however these systems are associated to both monetary and mobility cost.

Kuziek et al. propose in their article (“Transitioning EEG experiments away from the laboratory using a Raspberry Pi 2”. Journal of neuroscience methods, 277 (2017), 75-82) a stimuli presentation using a Rasperry Pi 2 computer to provide a small size and low cost alternative to desktop PC in the administration of cognitive stimulation. However, such a system showed high variability in trigger to stimulus onset timing quality which interferes with the amplitude and measurements of earlier event-related potentials. This variability in trigger to stimulus were related by the author to the slow processor of the Rasperry Pi 2 and the type of operational system running on it.

In this context, the invention herein described proposes a small size and low cost solution allowing to present a variety of highly controlled and accurate sensory stimulation and at the same time gather highly stable trigger to stimulus onset timing.

### SUMMARY

The present invention relates to a system for automatic evaluation of cognition and consciousness of a subject, said system comprising a micro-controller, a digital-to-analog converter and at least one sensory stimulation element, wherein the system is configured to receive as input an information concerning a stimulation paradigm and to generate as output a sensory stimulation according to the stimulation paradigm and transmit at least one synchronization signal determined by the stimulation paradigm to a device for the acquisition of a physiological signal so as to synchronize the system for stimulation with the device for the acquisition of a physiological signal during an acquisition. Advantageously the use of a micro-controller, which does not host an operating system, allows faster calculation and therefore a better synchronization between the system for stimulation and the device for the acquisition of a physiological signal.

According to one embodiment, the micro-controller is configured to receive as input the information concerning the stimulation paradigm and to generate a corresponding stimulation digital output.

According to one embodiment, the micro-controller is further configured to generate as digital output the synchronization signal which is transmitted, through a physical support, to the device for the acquisition of a physiological signal.

According to one embodiment, the synchronization signal is a temporal tag configured to time-locking an onset of at least one stimulus of the sensory stimulation with the related physiological signal.

According to one embodiment, the device for the acquisition of a physiological signal is an electroencephalogram device, an electrocardiogram device, a respiratory monitoring device, pupillometry device or a skin conductance measuring device.

According to one embodiment, the digital-to-analog converter is configured to receive the stimulation digital output and generate the sensory stimulation according to the stimulation paradigm by means of the sensory stimulation element.

According to one embodiment, the sensory stimulation element is configured to provide auditory stimulation, visual stimulation and/or haptic stimulation.

The system of the present invention advantageously allows to test the patients' responses using multiple sensory stimulation modalities. This represents a double advantage, first it allows using an appropriate modality when the patient has a sensory impairment. But more importantly, it allows to test the integration of multimodal sensory information in patients which, by itself, represents a hallmark of conscious processing.

According to one embodiment, the micro-controller is pre-programmed and programmable.

According to one embodiment, the information concerning the stimulation paradigm comprises information concerning the type of stimulation paradigm and at least one stimulation paradigm parameter.

According to one embodiment, the system comprises a user interface, notably a screen touch, through which the user selects the stimulation paradigm to be generated.

According to one embodiment, the system further comprises a proprietary non-volatile memory.

According to one embodiment, the stimulation digital output of the microcontroller is received by an external stimulation device, notably an electrical stimulation device configured to deliver somatosensory stimulation.

Yet another aspect of the present invention relates to method for the stimulation of a subject for cognitive evaluation comprising the steps of:
receiving clinical information concerning the patient;
receiving a selection of at least one stimulation paradigm obtained on the basis of the clinical information concerning the patient;
transmitting to the system according to any one of the embodiments described hereabove an input comprising information concerning the selected stimulation paradigm so that the system generates a sensory stimulation according to said stimulation paradigm and at least one synchronization signal determined by the stimulation paradigm;
transmitting the synchronization signal to a device for the acquisition of a physiological signal acquiring the physiological signal of the patient during the generation of the stimulation;
transferring the information concerning the selected stimulation paradigm, the physiological signal acquired and/or the synchronization signal to a database for storage.

According to one embodiment, the method of the present invention is a computer implemented method.

According to one embodiment, the method further comprises a step of analyzing the physiological signal acquired and the synchronization signal.

According to one embodiment, the physiological signal is an electrocardiographic signal, an electroencephalographic signal, a respiratory rate, an electrodermal activity signal and/or a pupil diameter measurement.

According to one embodiment, the clinical information is provided by a clinical database or by a user, wherein the clinical information provided by the user is further saved in the clinical database.

According to one embodiment, the method further comprises a step of generating a report comprising the results of the analysis step and the information concerning the stimulation paradigm selected.

The invention further relates to a computer program comprising instructions, which when the program is executed by a computer, causes the computer to carry out the steps of the method according to any one of the embodiments described hereabove.

The invention further relates to a computer-readable storage medium comprising instructions that when executed by a computer, causes the computer to carry out the steps of the method according to any one of the embodiments described hereabove.

The principal advantages of the present inventions are the capacity to unravel cognitive processes of non-communicating patients, regarding the temporal resolution, active paradigms, the easiness use at bedside in heavy ICU patients, comparing to other neuro-imagery techniques such as MRI and the possibility to have a longitudinal follow-up with repeated assessment thanks to the system that is configured to be put in the patient room.

### DEFINITIONS

In the present invention, the following terms have the following meanings:
“Electrode” refers to a conductor used to establish electrical contact with a nonmetallic part of a circuit, preferably a subject body. For instance, EEG electrodes are small metal discs usually made of stainless steel, tin, gold, silver covered with a silver chloride coating; there are placed on the scalp at specific positions.
“Electrocardiographic signal” refers to the signal recording the electrical conduction in the heart. Said cardiac signal may be for instance an electrocardiogram (EKG or ECG) or an endocardiogram. Such signals may have one or more channels, called leads. It may be short term (10 seconds in standard EKGs) or long term (several days in Holters).
“Electroencephalogram” or “EEG” refers to the tracing of brain waves, by recording the electrical activity of the brain from the scalp, made by an electroencephalograph.
“Electroencephalograph” refers to an apparatus for amplifying and recording brain waves.
“Mismatch negativity (MMN)” refers to a brain response to violations of a rule, established by a sequence of sensory stimuli (typically in the auditory domain). The mismatch negativity reflects the brain's ability to perform automatic comparisons between consecutive stimuli and provides an electrophysiological index of sensory learning and perceptual accuracy.
“Micro-controller” refers to a compact integrated circuit designed to govern a specific operation in an embedded system. A typical microcontroller includes a processor, memory and input/output peripherals on a single chip.
“P300 wave” refers to event related potential (ERP) component elicited in the process of decision making. More specifically, the P300 is thought to reflect processes involved in stimulus evaluation or categorization.
“Subject” refers to a mammal, preferably a human. In the sense of the present invention, a subject may be a patient, i.e. a person receiving medical attention, undergoing or having underwent a medical treatment, or monitored for the development of a disease.

### BRIEF DESCRIPTION OF THE DRAWINGS

Features and advantages of the invention will become apparent from the following description of embodiments of a system and a method for stimulation of a subject according to the invention, this description being given merely by way of example and with reference to the appended drawings in which:

FIG. 1 is a schematic representation of the system for automatic evaluation of cognition and consciousness of a subject according to one embodiment.

FIG. 2 is a block diagram of the steps implemented by the method for stimulation of a subject for cognitive evaluation according to one embodiment.

### DETAILED DESCRIPTION

The present invention relates to a system for automatic evaluation of cognition and consciousness of a subject. The system comprises at least one micro-controller, one digital-to-analog converter and at least one sensory stimulation element. The system is configured to receive as input an information concerning a stimulation paradigm and to generate as output a sensory stimulation according to the stimulation paradigm. In one embodiment, the stimulation paradigm comprises at least one pattern of sensory stimuli configured to induce a cognitive process in the stimulated subject.

Typically, a micro-controller (MCU) uses on-chip embedded Flash memory in which to store and execute its program without an operating system. Storing the program this way means the MCU having a shorter start-up period and executing code quickly. Without an operating system, a micro-controller can only run one control loop at a time. From a software perspective, this means a single thread is running on the microcontroller's processor or Central Processing Unit (CPU). The purpose of an MCU is to control electronic devices, usually running a fairly simple control loop forever or until it breaks or otherwise stops. A micro-controller is indeed different from a micro-processor in that, for example, a micro-processor will typically run an operating system. An operating system allows multiple processes to run at the same time via multiple threads, however performances of the micro-processor are directly dependent from the operating system.

Furthermore, as an MCU has all the components needed in its single chip, it does not need any external circuits to do its task so micro-controllers are heavily used in embedded systems. On the contrary, micro-processors have only a CPU inside them in one or few Integrated Circuits. Like micro-controllers it does not have RAM, ROM and other peripherals. They are dependent on external circuits of peripherals to work.

In the present invention the choice of using a micro-controller have been made due to the multiples aspects which differentiate it from a micro-processor, which is the main component of a standard computer.

In one embodiment, the system is further configured to transmit at least one synchronization signal determined by the stimulation paradigm to a device for the acquisition of a physiological signal so as to obtain a temporal synchronization between the system for stimulation and the device for the acquisition of a physiological signal during an acquisition. The temporal synchronization allows during the analysis of the physiological signal to identify the effect of the cognitive process induced by the sensory stimuli on the physiological activity under monitoring.

Temporal synchronization is indeed crucial for the estimation of the cognitive processes. In order to obtain a robust quantification of the brain activity triggered by the external stimuli, the latency between external stimuli and synchronization signal must be on the order of millisecond and very stable in time. The use of a micro-controller advantageously allows to guarantee a stable timing accuracy to the millisecond precision level for the visual stimuli and of the microsecond for the auditory stimuli. On the contrary, a computer (i.e. micro-processor) must coordinate with an operating system and external devices to continually have control of the computer's execution, the operating system or other devices can take control at any time. This will cause a sequence of stimuli to be delayed or suspended while the operating system task is completed. Furthermore, the nature and amount of delay depend from the type of micro-processor and the type of operating system. Therefore, even though computers may indeed implement a method consisting in:
receiving a stimulation paradigm; generating a sensory stimulation according to said stimulation paradigm and at least one synchronization signal determined by the stimulation paradigm;
transmitting the synchronization signal to a device for the acquisition of a physiological signal acquiring the physiological signal of the patient during the generation of the stimulation; and
transferring the information concerning the selected stimulation paradigm, the physiological signal acquired and/or the synchronization signal to a database for storage,
   each computer will perform with its own timing causing a disturbing variability among data collected from different computers. Indeed, as shown in Table 1 (data retrieved the 7 Jul. 2020, from web site https://support.pstnet.com/hc-en-us/articles/360008833253) computer does not present homogeneous delay.

Actually, computer have to be coupled to additional extra-loop (involving photo or audio sensors providing a supplementary synchronization signal) to correct for this problem and resynchronize the time stamp of the acquired data with the real timing of the stimulation paradigm.

Using a same micro-controller advantageously allow to remove the problem of inter-computer variability and obtain a much higher precision.

According to one embodiment, the micro-controller is configured to receive as input the information concerning the stimulation paradigm and to generate a corresponding stimulation digital output.

According to one embodiment, the information concerning the stimulation paradigm comprises information concerning the type of stimulation paradigm and at least one stimulation paradigm parameter. A type of stimulation paradigm may be a ‘local-global’ paradigm such as an oddball paradigm. An auditory oddball paradigm is a paradigm in which a sequence of stimuli is presented at each stimulation trial. The sequence is composed of a standard stimulus repeated a certain number of times, followed by a deviant stimulus. The comparison to a condition in which all the stimuli of the sequence are standard typically reveals the occurrence of the mismatch negativity. Crucially, if the deviant sequence is highly frequent within a block, the subjects would expect the last stimulus to be deviant. The sequence is thus standard at the global level (i.e. over the experimental block) and deviant at the local level (i.e. within a single trial). The local standard sequence becomes a global deviant and triggers the occurrence of a P300 component. The classic auditory oddball paradigm can be modified to produce different neural responses and can therefore be used to investigate dysfunctions in sensory and cognitive processing in clinical samples. The stimulation paradigm parameters that may be used are the time separating each stimulation trial or the time period in between the stimuli in one trial. The information concerning the stimulation paradigm may be selected by the user, notably health workers.

According to one embodiment, the system is configured to be remotely updated with information concerning at least one new stimulation paradigm. Thanks to this advantageous feature, the present invention is a flexible system wherein the number of available protocols (i.e. stimulation paradigm) is not fixed but may be added during time, for example by simple step of download of the new protocol.

According to one embodiment, the system further comprises a user interface, notably a screen touch, through which the user selects the stimulation paradigm to be generated. In one example, the display is a Gameduino 3 (FT810 GPU).

In one embodiment, the user interface is configured to display a menu to select various options concerning the type of stimulation paradigm and the stimulation paradigm parameter. In one example, the user interface is also configured to provide the status of the device and any related information such as battery charge information.

According to one embodiment, the micro-controller is further configured to generate as digital output the synchronization signal which is transmitted, through a physical support, to the device for the acquisition of a physiological signal.

According to one embodiment, the synchronization signal is a temporal tag configured to time-locking an onset of at least one stimulus of the sensory stimulation with the related physiological signal.

According to one embodiment, the micro-controller is used as embedded system in the system for automatic evaluation of cognition and consciousness of a subject.

According to one embodiment, the micro-controller has minimal requirements for memory and program length, with no operating system, and low software complexity.

In one embodiment, the micro-controller is an Advanced RISC Machine (ARM), which is a family of reduced instruction set computing (RISC) architectures for computer processors. Processors using a RISC architecture typically require fewer transistors than those with a complex instruction set computing (CISC) architecture (such as the x86 processors found in most personal computers), which improves cost, power consumption, and heat dissipation. The use of an ARM micro-controller advantageously to obtain a system which is light, portable and battery-powered.

According to one embodiment, the micro-controller is pre-programmed and programmable. More specifically, the micro-controller has an available on-chip memory, configured to fit the microcontroller program, which may be permanent, read-only memory that can only be programmed at the factory, or it may be field-alterable flash or erasable read-only memory.

According to one embodiment, the micro-controller is an Arduino Due which is microcontroller board based on Atmel SAM3X8E, 32-Bit ARM microcontroller. It contains 54 digital pins that can work both ways: input or output. Out of these digital pins, 12 can be used to generate pulse width modulation (PWM) outputs. Arduino Due module contains everything in bulk required for the automation project including 12 analog inputs. Arduino modules as the advantage of comprising built-in peripherals and an ability to perform a number of functions on a single chip. Also, no external burner is required for Arduino modules, as it comes with a built-in burner. Furthermore, Arduino Due advantageously operates at low voltages around 3.3V. The low power consumption of the chosen micro-controller allows the system to run on rechargeable batteries advantageously allowing to reduce electrical interference with the device for the acquisition of a physiological signal.

According to one embodiment, the digital-to-analog converter is configured to receive and convert the stimulation digital output, i.e. the digital data produced as output by the micro-controller, into an analog signal to be transmitted to the sensory stimulation element so as to generate the sensory stimulation according to the stimulation paradigm by means of the sensory stimulation element.

According to one embodiment, the digital-to-analog converter of the present invention is a 2-channel low power DAC (Digital-to-Analog Converter). The low power consumption allows the system to run on rechargeable batteries advantageously allowing to reduce electrical interference with the device for the acquisition of a physiological signal.

According to one embodiment, the sensory stimulation element is configured to provide auditory stimulation, visual stimulation and/or haptic stimulation. In one example, the patient may be stimulate using only one kind of sensory stimulation like the auditory stimulation or he/she may be stimulated simultaneously or consecutively with different kind of sensory stimulations. The use of multiple sensory modalities (auditory, visual, tactile, cross-modal) allows to achieve a high sensitivity to detect conscious perceptual processing. This advantageously allows to implement a full multimodal neurophysiological prognosis assessment procedure.

In one embodiment, the sensory stimulation element for visual stimulation comprises at least two LED matrix allowing to display custom visual stimuli for each visual field independently. In one example, the LED matrix comprises three color LED arranged in an 8×8 matrix.

In one embodiment, the sensory stimulation element for haptic stimulation comprises two haptic motor controllers capable of producing more than 100 different effects and the possibility of produce customize effects.

According to one embodiment, the stimulation digital output of the microcontroller is received by an external stimulation device, notably an electrical stimulation device configured to deliver somatosensory stimulation. In one example, an independent CE-marked electrical stimulator can be used.

In one example, the sensory stimulation element is configured to produce auditory stimulation, notably it is a stereo configured to reproduce WAV-files. The digital-to-analog converter is a 2-channel low power audio DAC, such as a UDA1334ATS. In this example, the system further comprises an audio amplifier in a custom setup (LM4808M dual audio power amplifier capable of delivering 105 mW per channel).

According to one embodiment, the system further comprises a Network Communication configured to allow accessing a central server through a wired or wireless TCP/IP network (internet/intranet). In this embodiment, the Network Communication is used to indicate to the central server that a new stimulation will take place, indicating starting time, protocol (i.e. the type of stimulation paradigm and at least one stimulation paradigm parameter) and other relevant information. Alternatively, the Network Communication may be also used to update the available stimulation paradigm on the system by downloading updated information from the server.

According to one embodiment, the device for the acquisition of a physiological signal is an electroencephalogram device.

According to one embodiment, the electroencephalogram device comprises a plurality of electrodes, positioned onto predetermined areas of the scalp of the subject in order to obtain a multi-channel electroencephalographic signal. According to one embodiment, the electroencephalogram device is configured to acquire electroencephalographic signals from at least 4, 8, 10, 15, 16, 17, 18, 19, 20, 21, 32, 64, 128 or 256 electrodes. According to one embodiment, the electrodes are placed on the scalp according to the 10-10 or 10-20 system, dense-array positioning or any other electrodes positioning known by the man skilled in the art. The electrodes montage may be unipolar or bipolar. In a preferred embodiment, the electrodes are placed accordingly to the 10-20 system in a bipolar montage. In one embodiment, the plurality of electrodes are dry electrodes or semi-dry electrodes. Electrode material may be a metal such as stainless steel or copper, such as inert metals, like, gold, silver (silver/silver chloride), tin and the like. The electrodes may be flexible, pre-shaped or rigid, and in any shape, for example, a sheet, rectangular, circular, or such other shape conducive to make contact with the wearer's skin. In a preferred embodiment, the electrodes are textile electrodes. In said embodiment, various types of suitable headsets or electrode systems are available for acquiring such neural signals. The possibility of connecting the system to a variety of different clinical EEG recording devices allows to expand the implantation scope of the present system to different technological configurations that exist in different hospitals and clinics.

According to one embodiment, the device for the acquisition of a physiological signal is an electrocardiogram device. According to one embodiment, the electrocardiogram device is a standard 12 leads device.

According to one embodiment, the device for the acquisition of a physiological signal is configured to measure a respiratory signal, such as the respiratory rate. The respiratory rate may be measured using a fiber-optic breath rate sensor, impedance pneumography or capnography which are commonly implemented in patient monitoring. The respiratory rate may be as well estimated indirectly from measurements performed with electrocardiogram, photoplethysmogram and accelerometry signals.

According to one embodiment, the device for the acquisition of a physiological signal is configured to measure pupil diameter and reactivity (i.e. pupillometry device).

According to one embodiment, the device for the acquisition of a physiological signal is configured to measure electrodermal activity (EDA), also known as skin conductance, which is the property of the human body that causes continuous variation in the electrical characteristics of the skin. Skin conductance is not under conscious control but is modulated autonomously by sympathetic activity which drives human behavior, cognitive and emotional states on a subconscious level. Skin conductance advantageously offers direct insights into autonomous emotional regulation.

According to one embodiment, the system further comprises a proprietary non-volatile memory controller and memory, notably a MicroSD Controller such as Adafruit MicroSD card breakout board with a generic MicroSD card.

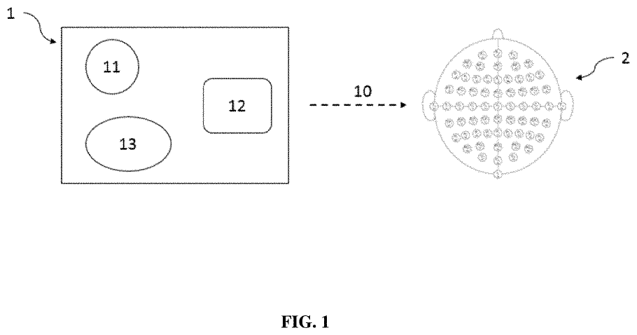

FIG. 1 provides a schematic representation of one embodiment of the system 1, comprising a micro-controller 11, a digital-to-analog converter 12 and at least one sensory stimulation element 13. In this illustrated embodiment, the synchronization signal 10 determined by the stimulation paradigm to an electroencephalogram device being the device for the acquisition of a physiological signal 2.

The present invention further relates to a method for stimulation of a subject for cognitive evaluation which comprises multiple steps.

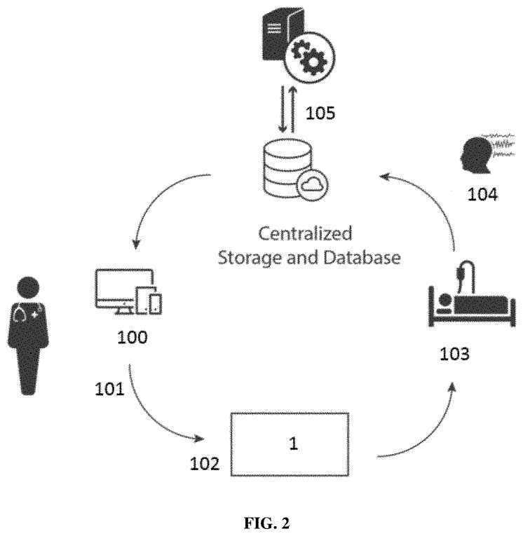

As illustrated in the block diagram in FIG. 2, according to one embodiment, the first step of the method consists in the reception of clinical information concerning the patient 100.

According to one embodiment, the clinical information is provided by a clinical database or by a user, wherein the clinical information provided by the user is further saved in the clinical database. The clinical information may be visualized by the user, notably a health worker, on a display.

According to one embodiment, the method further comprises a step of selecting of at least one stimulation paradigm obtained on the basis of the clinical information concerning the patient 101. In one example, the stimulation paradigm is chosen by the user on the bases of the clinical information previously visualized.

In the one embodiment, the method further comprises a step of transmitting to the system according to any one of the embodiments described hereabove an input comprising information concerning the selected stimulation paradigm 102 so that the system generates a sensory stimulation according to said stimulation paradigm and at least one synchronization signal determined by the stimulation paradigm. The information may be selected on a computer on which the user had visualized the clinical information and transmitted to the system wirelessly or through a physical cable. Alternatively, the information may be transmitted to the system directly by the used by selecting directly on the screen touch of the system the desired stimulation paradigm.

In the one embodiment, the method further comprises a step of transmitting the synchronization signal to a device for the acquisition of a physiological signal acquiring the physiological signal of the patient during the generation of the stimulation 103. In one embodiment, the synchronization signal is transmitted through a physical support, such as a cable.

In the one embodiment, the method further comprises a step of transferring the information concerning the selected stimulation paradigm, the physiological signal acquired and/or the synchronization signal to a database for storage 104.

According to one embodiment, the method comprises a step of analyzing the physiological signal acquired and the synchronization signal 105. According to one embodiment, the analysis step 105 is configured so as to allow the analysis of any type of signal produced by currently available devices for the acquisition of a physiological signals (for example EEG devices from different manufactures).

In one embodiment, the analysis of the physiological signal is configured to estimate the level of consciousness in patients recovering from coma and other related disorders. The present method advantageously provides to the clinician and caregivers with detailed information about the patients' current and future state of consciousness. According to one embodiment, the physiological signal is an electrocardiographic signal, an electroencephalographic signal, a respiratory rate and/or a pupil diameter measurement.

The evaluation of consciousness and cognitive abilities through analysis of physiological signals according to the method of the present may be used for the diagnosis of the following brain disorders: brain anoxia (e.g.: after cardiac arrest); traumatic brain injury; ischemic or hemorrhagic stroke; metabolic encephalopathy, encephalitis or meningoencephalitis, neurodegenerative disorders (including Alzheimer diseased and related diseases) and neurodevelopmental disorders (including ADHD, dyspraxia, dysphasia and related syndromes).

According to one embodiment, the method comprises a step of generating a report comprising the results of the analysis step and the information concerning the stimulation paradigm selected. In particular, the report is transmitted to the user (i.e. the clinician and caregivers) for review. The report advantageously provides a feedback from the results of the EEG analysis to allow the user to launch a more appropriated stimulation protocol. This means adapting the parameters of the stimulation or even playing a more specific cognitive stimulation to better evaluate the true cognitive state of the patient.

Furthermore, the analysis step 105 may be performed directly by the system of the present invention or alternatively the physiological signal acquired and the synchronization signal may be transferred to a remote server where clinicians can upload their data in a simple manner and receive, within minutes, the report comprising the results of the analysis step and the information concerning the stimulation paradigm selected. In one embodiment, the server is a web-server. According to one embodiment, the web-server provides an HTML-report that includes all values, statistics, images, and interactive elements obtained by the analysis step 105 in a single file that can be conveniently sent to users and reviewed in their own browser.

While various embodiments have been described and illustrated, the detailed description is not to be construed as being limited hereto. Various modifications can be made to the embodiments by those skilled in the art without departing from the true spirit and scope of the disclosure as defined by the claims.

### REFERENCES

1—System;

2—Device for the acquisition of a physiological signal;

10—Synchronization signal;

11—Micro-controller;

12—Digital-to-analog converter;

13—Sensory stimulation element;

100—Step of receiving clinical information concerning the patient;

101—Step of receiving a selection of at least one stimulation paradigm obtained on the basis of the clinical information concerning the patient;

102—Step of transmitting to the system an input comprising information concerning the selected stimulation paradigm;

103—Step of transmitting the synchronization signal to a device for the acquisition of a physiological signal;

104—Step of transferring the information to a database for storage;

105—Step of analyzing the physiological signal acquired and the synchronization signal

1-15. (canceled)

16. A system for automatic evaluation of cognition and consciousness of a subject through external stimulations, said system comprising a micro-controller, a digital-to-analog converter and at least one sensory stimulation element, wherein the system is configured to receive as input an information concerning a stimulation paradigm and to generate as output a sensory stimulation according to the stimulation paradigm and transmit at least one synchronization signal determined by the stimulation paradigm to a device for the acquisition of a physiological signal so as to synchronize the system for stimulation with the device for the acquisition of a physiological signal during an acquisition.

17. The system according to claim 16, wherein the micro-controller is configured to receive as input the information concerning the stimulation paradigm and to generate a corresponding stimulation digital output.

18. The system according to claim 16, wherein the micro-controller is further configured to generate as digital output the synchronization signal which is transmitted, through a physical support, to the device for the acquisition of a physiological signal.

19. The system according to claim 16, wherein the synchronization signal is a temporal tag configured to time-locking an onset of at least one stimulus of the sensory stimulation with the related physiological signal.

20. The system according to claim 16, wherein the device for the acquisition of a physiological signal is an electroencephalogram device or an electrocardiogram device.

21. The system according to claim 16, wherein the digital-to-analog converter is configured to receive the stimulation digital output and generate the sensory stimulation according to the stimulation paradigm by means of the sensory stimulation element.

22. The system according to claim 16, wherein the sensory stimulation element is configured to provide auditory stimulation, visual stimulation and/or haptic stimulation.

23. The system according to claim 16, wherein the micro-controller is pre-programmed and programmable.

24. The system according to claim 17, wherein the information concerning the stimulation paradigm comprises information concerning the type of stimulation paradigm and at least one stimulation paradigm parameter.

25. The system according to claim 16, further comprising a proprietary non-volatile memory and a user interface, notably a screen touch, through which the user selects the stimulation paradigm to be generated.

26. The system according to claim 16, wherein the stimulation digital output of the microcontroller is received by an external stimulation device, notably an electrical stimulation device configured to deliver somatosensory stimulation.

27. A method for the stimulation of a subject for cognitive evaluation comprising the steps of:
receiving clinical information concerning the patient;
receiving a selection of at least one stimulation paradigm obtained on the basis of the clinical information concerning the patient;
transmitting to the system according to claim 16 an input comprising information concerning the selected stimulation paradigm so that the system generates a sensory stimulation according to said stimulation paradigm and at least one synchronization signal determined by the stimulation paradigm;
transmitting the synchronization signal to a device for the acquisition of a physiological signal acquiring the physiological signal of the patient during the generation of the stimulation; and
transferring the information concerning the selected stimulation paradigm, the physiological signal acquired and/or the synchronization signal to a database for storage.

28. The method according to claim 27, further comprises a step of analyzing the physiological signal acquired and the synchronization signal.

29. The method according to claim 27, wherein the clinical information is provided by a clinical database or by a user, wherein the clinical information provided by the user is further saved in the clinical database.

30. The method according to claim 27, further comprising a step of generating a report comprising the results of the analysis step and the information concerning the stimulation paradigm selected.
